(19) United States
(12) Patent Application Publication
(10) Pub. No.: US 2026/0283787 A1
(43) Pub. Date: Sep. 24, 2026

(54) LENSES HAVING DIFFRACTIVE PROFILES WITH IRREGULAR WIDTH FOR VISION TREATMENT

(71) Applicant: AMO Groningen B.V., Groningen (NL)

(72) Inventors: Robert Rosen, Groningen (NL); Franck Gounou, Groningen (NL)

(21) Appl. No.: 19/685,031

(22) Filed: May 21, 2026

Related U.S. Application Data
(62) Division of application No. 17/103,944, filed on Nov. 24, 2020, now Pat. No. 12,653,665.
(60) Provisional application No. 62/955,355, filed on Dec. 30, 2019.

(51) Int. Cl. A61F 2/16; B29D 11/00; G16H 20/30

(52) U.S. Cl. CPC A61F 2/1656; A61F 2/1624; A61F 2/1637; A61F 2/1654; B29D 11/00269; B29D 11/00961; G16H 20/30; A61F 2240/002; G05B 2219/2617

(57) ABSTRACT

Apparatuses, systems and methods for providing improved ophthalmic lenses, particularly intraocular lenses (IOLs), include features for providing improved extended depth of focus lenses. Exemplary ophthalmic lenses can include an optic including a diffractive profile including a plurality of echelettes, each echelette of the diffractive profile having a different width in r-squared space than any other echelette of the diffractive profile and each echelette of the diffractive profile being configured to distribute light to a distance focus.

### CROSS REFERENCE TO RELATED APPLICATIONS

This application is a division of U.S. patent application Ser. No. 17/103,944, filed Nov. 24, 2020, which claims priority to U.S. Provisional Patent Application No. 62/955355, filed on Dec. 30, 2019, the entire contents of which are hereby incorporated by reference.

### BACKGROUND

Embodiments of the present disclosure relate to vision treatment techniques and in particular, to ophthalmic lenses such as, for example, contact lenses, corneal inlays or onlays, or intraocular lenses (IOLs) including, for example, phakic IOLs and piggyback IOLs (i.e. IOLs implanted in an eye already having an IOL).

Presbyopia is a condition that affects the accommodation properties of the eye. As objects move closer to a young, properly functioning eye, the effects of ciliary muscle contraction and zonular relaxation allow the lens of the eye to change shape, and thus increase its optical power and ability to focus at near distances. This accommodation can allow the eye to focus and refocus between near and far objects.

Presbyopia normally develops as a person ages and is associated with a natural progressive loss of accommodation. The presbyopic eye often loses the ability to rapidly and easily refocus on objects at varying distances. The effects of presbyopia usually become noticeable after the age of 45 years. By the age of 65 years, the crystalline lens has often lost almost all elastic properties and has only a limited ability to change shape.

Along with reductions in accommodation of the eye, age may also induce clouding of the lens due to the formation of a cataract. A cataract may form in the hard central nucleus of the lens, in the softer peripheral cortical portion of the lens, or at the back of the lens. Cataracts can be treated by the replacement of the cloudy natural lens with an artificial lens. An artificial lens replaces the natural lens in the eye, with the artificial lens often being referred to as an intraocular lens or “IOL.”

Monofocal IOLs are intended to provide vision correction at one distance only, usually the far focus. At the very least, since a monofocal IOL provides vision treatment at only one distance and since the typical correction is for far distance, spectacles are usually needed for good vision at near distances and sometimes for good vision at intermediate distances. The term “near vision” generally corresponds to vision provided when objects are at a distance from the subject eye at equal; or less than 1.5 feet. The term “distant vision” generally corresponds to vision provided when objects are at a distance of at least about 5-6 feet or greater. The term “intermediate vision” corresponds to vision provided when objects are at a distance of about 1.5 feet to about 5-6 feet from the subject eye. Such characterizations of near, intermediate, and far vision correspond to those addressed in Morlock R, Wirth RJ, Tally SR, Garufis C, Heichel CWD, Patient-Reported Spectacle Independence Questionnaire (PRSIQ): Development and Validation. Am J Ophthalmology 2017; 178:101-114.

There have been various attempts to address limitations associated with monofocal IOLs. For example, multifocal IOLs have been proposed that deliver, in principle, two foci, one near and one far, optionally with some degree of intermediate focus. Such multifocal, or bifocal, IOLs are intended to provide good vision at two distances, and include both refractive and diffractive multifocal IOLs. In some instances, a multifocal IOL intended to correct vision at two distances may provide a near (add) power of about 3.0 or 4.0 diopters.

Multifocal IOLs may, for example, rely on a diffractive optical surface to direct portions of the light energy toward differing focal distances, thereby allowing the patient to clearly see both near and far objects. Multifocal ophthalmic lenses (including contact lenses or the like) have also been proposed for treatment of presbyopia without removal of the natural crystalline lens. Diffractive optical surfaces, either monofocal or multifocal, may also be configured to provide reduced chromatic aberration.

Diffractive monofocal and multifocal lenses can make use of a material having a given refractive index and a surface curvature which provide a refractive power. Diffractive lenses have a diffractive profile which confers the lens with a diffractive power that contributes to the overall optical power of the lens. The diffractive profile is typically characterized by a number of diffractive zones. When used for ophthalmic lenses these zones are typically annular lens zones, or echelettes, spaced about the optical axis of the lens. Each echelette may be defined by an optical zone, a transition zone between the optical zone and an optical zone of an adjacent echelette, and an echelette geometry. The echelette geometry includes an inner and outer diameter and a shape or slope of the optical zone, a height or step height, and a shape of the transition zone. The surface area or diameter of the echelettes largely determines the diffractive power(s) of the lens and the step height of the transition between echelettes largely determines the light distribution between the different powers. Together, these echelettes form a diffractive profile.

A multifocal diffractive profile of the lens may be used to mitigate presbyopia by providing two or more optical powers; for example, one for near vision and one for far vision. The lenses may also take the form of an intraocular lens placed within the capsular bag of the eye, replacing the original lens, or placed in front of the natural crystalline lens. The lenses may also be in the form of a contact lens, most commonly a bifocal contact lens, or in any other form mentioned herein.

Although multifocal ophthalmic lenses lead to improved quality of vision for many patients, additional improvements would be beneficial. For example, some pseudophakic patients experience undesirable visual effects (dysphotopsia), e.g. glare or halos. Halos may arise when light from the unused focal image creates an out-of-focus image that is superimposed on the used focal image. For example, if light from a distant point source is imaged onto the retina by the distant focus of a bifocal IOL, the near focus of the IOL will simultaneously superimpose a defocused image on top of the image formed by the distant focus. This defocused image may manifest itself in the form of a ring of light surrounding the in-focus image, and is referred to as a halo. Another area of improvement revolves around the typical bifocality of multifocal lenses. While multifocal ophthalmic lenses typically provide adequate near and far vision, intermediate vision may be compromised.

A lens with an extended range of vision may thus provide certain patients the benefits of good vision at a range of distances, while having reduced or no dysphotopsia. Various techniques for extending the depth of focus of an IOL have been proposed. One technique is embodied in the Tecnis Symfony® lens offered by Johnson & Johnson Vision. One technique may include a bulls-eye refractive principle, and may involve a central zone with a slightly increased power. One technique may include an asphere or include refractive zones with different refractive zonal powers.

Although certain proposed treatments may provide some benefit to patients in need thereof, further advances would be desirable. For example, it would be desirable to provide improved IOL systems and methods that confer enhanced image quality across a wide and extended range of foci without dysphotopsia. Embodiments of the present disclosure provide solutions that address the problems described above, and hence provide answers to at least some of these outstanding needs.

### BRIEF SUMMARY

Embodiments herein described include ophthalmic lenses including an optic. The optic may include a diffractive profile including a plurality of echelettes, each echelette of the diffractive profile having a different width in r-squared space than any other echelette of the diffractive profile and each echelette of the diffractive profile being configured to distribute light to a distance focus.

The plurality of echelettes may include at least 3 echelettes, at least 4 echelettes, or at least 5 echelettes. Each echelette of the diffractive profile may be configured to distribute light to a near focus. At least two of the echelettes of the diffractive profile may have a different step height. It is also envisioned that at least two of the echelettes of the diffractive profile may have a same step height. The optic may include a central zone and a peripheral zone, and the diffractive profile may be positioned on the central zone with the peripheral zone including a refractive surface. The diffractive profile may form an extended depth of focus.

The optic may include a first surface and a second surface each disposed about an optical axis and extending radially outward from the optical axis to an outer periphery of the optic. The first surface faces opposite the second surface with a plurality of echelettes of a diffractive profile disposed on the first surface such that each one of the plurality of echelettes on the first surface between the optical axis and the outer periphery of the optic has a different width in r-squared space as opposed to any other echelette on the first surface between the optical axis and the outer periphery of the optic.

Embodiments herein described include a method comprising fabricating an optic for an ophthalmic lens, the optic including a diffractive profile including a plurality of echelettes, each echelette of the diffractive profile having a different width in r-squared space than any other echelette of the diffractive profile and each echelette of the diffractive profile being configured to distribute light to a distance focus. The method may further comprise receiving an ophthalmic lens prescription, and fabricating the optic based on the ophthalmic lens prescription. One or more of the diffractive profile or a refractive profile of the optic may be determined based on the ophthalmic lens prescription. This method of fabrication may be used to fabricate any lens disclosed herein.

Embodiments herein described include a system for fabricating an ophthalmic lens. The system may include a processor configured to determine a diffractive profile of an optic, the diffractive profile including a plurality of echelettes, each echelette of the diffractive profile having a different width in r-squared space than any other echelette of the diffractive profile and each echelette of the diffractive profile being configured to distribute light to a distance focus. The system may include a manufacturing assembly that fabricates the optic based on the diffractive profile. The method may further comprise an input for receiving an ophthalmic lens prescription, wherein the processor is configured to determine one or more of the diffractive profile or a refractive profile of the optic based on the ophthalmic lens prescription. This system for fabricating may be used to fabricate any lens disclosed herein.

### BRIEF DESCRIPTION OF THE DRAWINGS

FIG. 1A illustrates a cross-sectional view of an eye with an implanted multifocal refractive intraocular lens.

FIG. 1B illustrates a cross-sectional view of an eye having an implanted multifocal diffractive intraocular lens.

FIG. 2A illustrates a front view of a diffractive multifocal intraocular lens.

FIG. 2B illustrates a cross-sectional view of a diffractive multifocal intraocular lens.

FIG. 3A-3B are graphical representations of a portion of the diffractive profile of a conventional diffractive multifocal lens.

FIG. 4 illustrates a diffractive profile of a bifocal optic.

FIG. 5 illustrates a diffractive profile of a trifocal optic.

FIG. 6 illustrates a profile of an optic in which a trifocal diffractive pattern is only positioned on a central zone of an optic, with a peripheral zone including a refractive surface.

FIG. 7 illustrates a profile of an optic that does not include echelettes that repeat having the same width in r-squared space.

FIG. 8 illustrates an embodiment of a system.

### DETAILED DESCRIPTION

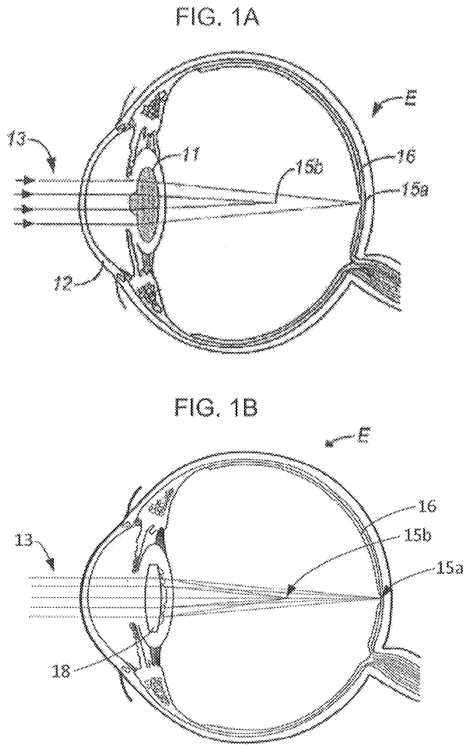

FIGS. 1A, 1B, 2A, 2B, 3A and 3B illustrate multifocal IOL lens geometries, aspects of which are described in U.S. Patent Publication No. 2011-0149236 A1, which is hereby incorporated by reference in its entirety.

FIG. 1A is a cross-sectional view of an eye E fit with a multifocal IOL 11. As shown, multifocal IOL 11 may, for example, comprise a bifocal IOL. Multifocal IOL 11 receives light from at least a portion of cornea 12 at the front of eye E and is generally centered about the optical axis of eye E. For ease of reference and clarity, FIGS. 1A and 1B do not disclose the refractive properties of other parts of the eye, such as the corneal surfaces. Only the refractive and/or diffractive properties of the multifocal IOL 11 are illustrated.

Each major face of lens 11, including the anterior (front) surface and posterior (back) surface, generally has a refractive profile, e.g. biconvex, plano-convex, plano-concave, meniscus, etc. The two surfaces together, in relation to the properties of the surrounding aqueous humor, cornea, and other optical components of the overall optical system, define the effects of the lens 11 on the imaging performance by eye E. Conventional, monofocal IOLs have a refractive power based on the refractive index of the material from which the lens is made, and also on the curvature or shape of the front and rear surfaces or faces of the lens. One or more support elements may be configured to secure the lens 11 to a patient's eye.

Multifocal lenses may optionally also make special use of the refractive properties of the lens. Such lenses generally include different powers in different regions of the lens so as to mitigate the effects of presbyopia. For example, as shown in FIG. 1A, a perimeter region of refractive multifocal lens 11 may have a power which is suitable for viewing at far viewing distances. The same refractive multifocal lens 11 may also include an inner region having a higher surface curvature and a generally higher overall power (sometimes referred to as a positive add power) suitable for viewing at near distances.

Rather than relying entirely on the refractive properties of the lens, multifocal diffractive IOLs or contact lenses can also have a diffractive power, as illustrated by the IOL 18 shown in FIG. 1B. The diffractive power can, for example, comprise positive or negative power, and that diffractive power may be a significant (or even the primary) contributor to the overall optical power of the lens. The diffractive power is conferred by a plurality of concentric diffractive zones which form a diffractive profile. The diffractive profile may either be imposed on the anterior face or posterior face or both.

The diffractive profile of a diffractive multifocal lens directs incoming light into a number of diffraction orders. As light 13 enters from the front of the eye, the multifocal lens 18 directs light 13 to form a far field focus 15a on retina 16 for viewing distant objects and a near field focus 15b for viewing objects close to the eye. Depending on the distance from the source of light 13, the focus on retina 16 may be the near field focus 15b instead. Typically, far field focus 15a is associated with 0^{th} diffractive order and near field focus 15b is associated with the 1^{st} diffractive order, although other orders may be used as well.

Bifocal ophthalmic lens 18 typically distributes the majority of light energy into two viewing orders, often with the goal of splitting imaging light energy about evenly (50%:50%), one viewing order corresponding to far vision and one viewing order corresponding to near vision, although typically, some fraction goes to non-viewing orders.

Corrective optics may be provided by phakic IOLs, which can be used to treat patients while leaving the natural lens in place. Phakic IOLs may be angle supported, iris supported, or sulcus supported. The phakic IOL can be placed over the natural crystalline lens or piggy-backed over another IOL. It is also envisioned that the present disclosure may be applied to inlays, onlays, accommodating IOLs, pseudophakic IOLs, other forms of intraocular implants, spectacles, and even laser vision correction.

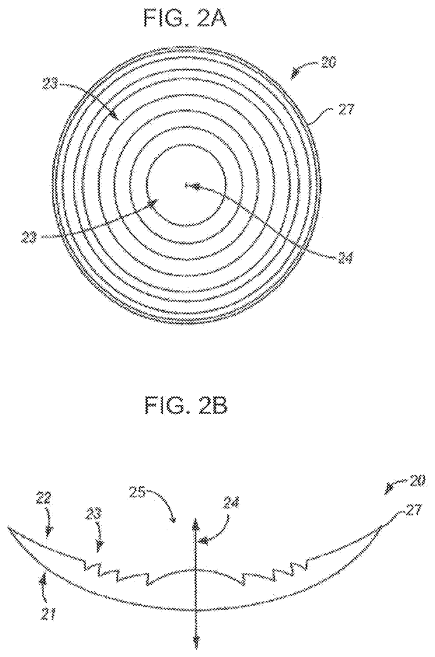

FIGS. 2A and 2B show aspects of a conventional diffractive multifocal lens 20. Multifocal lens 20 may have certain optical properties that are generally similar to those of multifocal IOLs 11, 18 described above. Multifocal lens 20 has an anterior lens face 21 and a posterior lens face 22 disposed about an optical axis 24. The faces 21, 22, or optical surfaces, extend radially outward from the optical axis 24 to an outer periphery 27 of the optic. The faces 21, 22, or optical surfaces, face opposite each other.

When fitted onto the eye of a subject or patient, the optical axis of lens 20 is generally aligned with the optical axis of eye E. The curvature of lens 20 gives lens 20 an anterior refractive profile and a posterior refractive profile. Although a diffractive profile may also be imposed on either anterior face 21 and posterior face 22 or both, FIG. 2B shows posterior face 22 with a diffractive profile. The diffractive profile is characterized by a plurality of annular diffractive zones or echelettes 23 spaced about optical axis 24. While analytical optics theory generally assumes an infinite number of echelettes, a standard multifocal diffractive IOL typically has at least 9 echelettes, and may have over 30 echelettes. For the sake of clarity, FIG. 2B shows only 4 echelettes. Typically, an IOL is biconvex, or possibly plano-convex, or convex-concave, although an IOL could be plano-plano, or other refractive surface combinations.

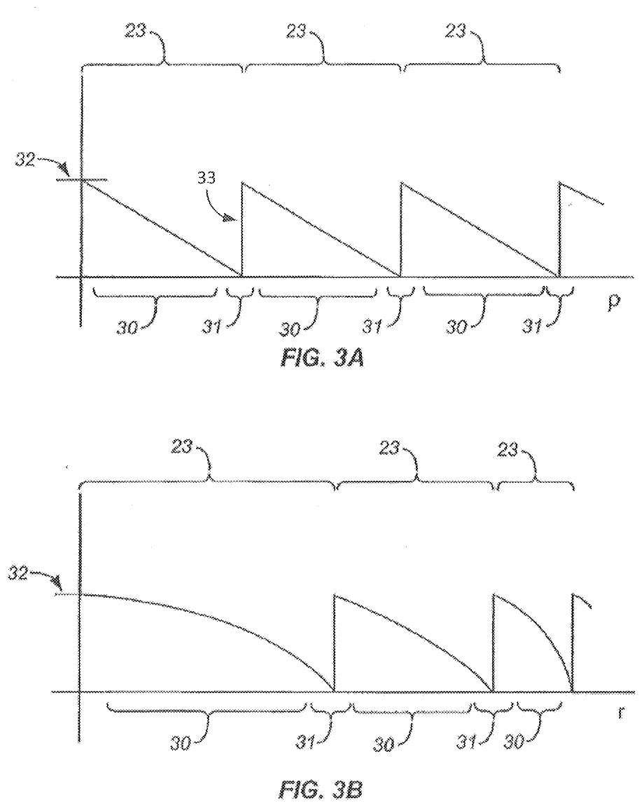

FIGS. 3A and 3B are graphical representations of a portion of a typical diffractive profile of a multifocal lens. While the graph shows only 3 echelettes, typical diffractive lenses extend to at least 9 echelettes to over 32 echelettes. In FIG. 3A, the height 32 of the surface relief profile (from a plane perpendicular to the light rays) of each point on the echelette surface is plotted against the square of the radial distance (r² or ρ) from the optical axis of the lens (referred to as r-squared space). In multifocal lenses, each echelette 23 may have a diameter or distance from the optical axis which is often proportional to √n, n being the number of the echelette 23 as counted from optical axis 24. Each echelette has a characteristic optical zone 30 and transition zone 31. Optical zone 30 typically has a shape or downward slope that is parabolic as shown in FIG. 3B. The slope of each echelette in r-squared space (shown in FIG. 3A), however, is the same. As for the typical diffractive multifocal lens, as shown here, all echelettes have the same surface area. The area of echelettes 23 determines the diffractive power of lens 20, and, as area and radii are correlated, the diffractive power is also related to the radii of the echelettes. The physical offset of the trailing edge of each echelette to the leading edge of the adjacent echelette is the step height. An exemplary step height between adjacent echelettes is marked as reference number 33 in FIG. 3A. The step heights remain the same in r-squared space (FIG. 3A) and in linear space (FIG. 3B). The step offset is the height offset of the transition zone from the underlying base curve. An exemplary step offset is marked as reference number 501 in FIG. 5.

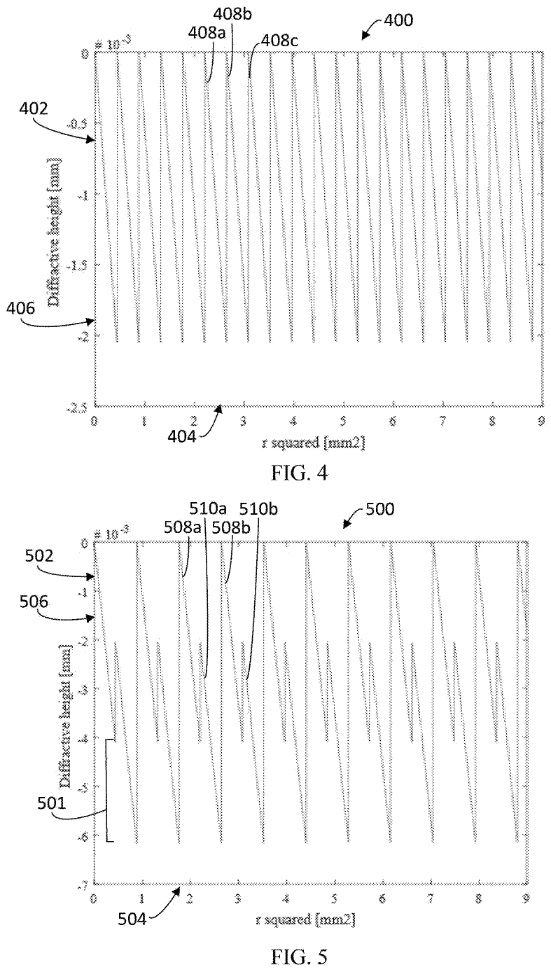

Conventional multifocal diffractive lenses typically provide for near and far vision, neglecting visual performance at intermediate distances. FIG. 4, for example, illustrates a diffractive profile of a bifocal optic. The diffractive profile 400 is shown relative to the Y axis 402, which represents the phase shift of the diffractive profile 400. The height is shown in units of millimeter (mm), and may represent the distance from the base spherical wavefront generated by the lens. In other embodiments, other units or scalings may be utilized. The height or phase shift of the diffractive profile 400 is shown in relation to the radius on the X axis 404 from the optical axis 406 in r-squared space. The radial coordinate represents the distance from the optical axis 406 in r-squared space, and is shown in units of millimeters squared, although in other embodiments, other units or scalings may be utilized.

The diffractive profile 400 includes a plurality of echelettes (representative echelettes 408a, 408b, 408c are marked) that each have the same width in r-squared space. The step height of each echelette is also the same in the diffractive profile 400. The diffractive pattern accordingly may direct light to two focuses (forming a bifocal optic).

FIG. 5 illustrates a diffractive profile of a trifocal optic. The diffractive profile 500 is shown relative to the Y axis 502, which represents the phase shift of the diffractive profile 500. The height is shown in units of millimeter (mm), and may represent the distance from the base spherical wavefront generated by the lens. In other embodiments, other units or scalings may be utilized. The height or phase shift of the diffractive profile 500 is shown in relation to the radius on the X axis 504 from the optical axis 506 in r-squared space. The radial coordinate represents the distance from the optical axis 506 in r-squared space, and is shown in units of millimeters squared, although in other embodiments, other units or scalings may be utilized.

The diffractive profile 500 includes a plurality of echelettes. The echelettes are provided as repeating set of two different echelettes, with one configuration of echelette being marked in FIG. 5 as echelettes 508a, 508b, and another configuration of echelette being marked in FIG. 5 as echelette 510a, 510b. The width in r-squared space and the step height and step offset of each configuration of echelette (e.g., echelette 508a, 508b) is repeated upon the optic. The echelettes 508a, 508b for example have the same width in r-squared space as each other, and the echelettes 510a, 510b have the same width in r-squared space as each other. The diffractive pattern accordingly may direct light to three focuses (forming a trifocal optic).

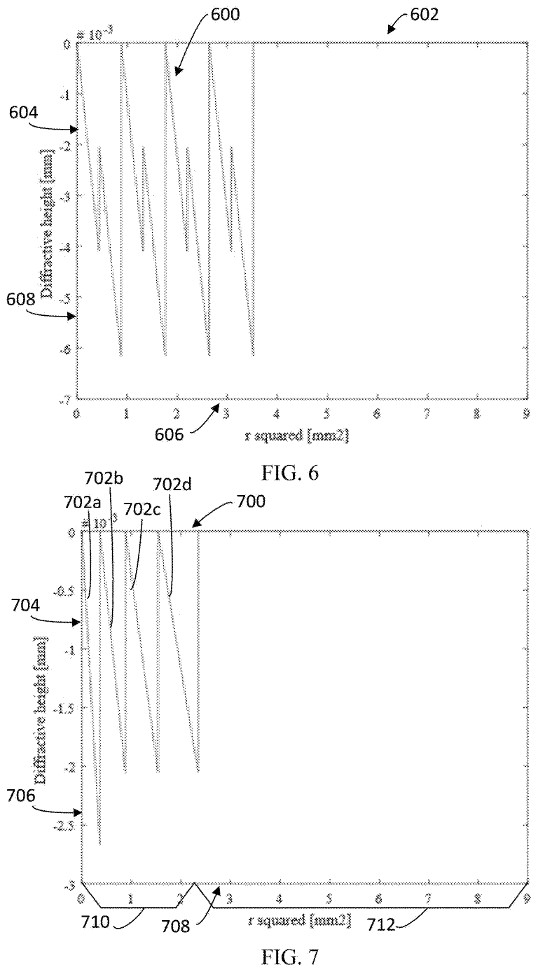

The optic represented in FIG. 5 may be modified to include at least one zone that does not include the diffractive pattern. FIG. 6 for example represents an optic in which a trifocal diffractive pattern 600 is only positioned on a central zone of an optic, with a peripheral zone including a refractive surface 602. In FIG. 6, the diffractive profile 600 is shown relative to the Y axis 604, which represents the phase shift of the diffractive profile 600. The height is shown in units of millimeters (mm), and may represent the distance from the base spherical wavefront generated by the lens. In other embodiments, other units or scalings may be utilized. The height or phase shift of the diffractive profile 600 is shown in relation to the radius on the X axis 606 from the optical axis 608 in r-squared space. The radial coordinate represents the distance from the optical axis 608 in r-squared space, and is shown in units of millimeters squared, although in other embodiments, other units or scalings may be utilized.

FIG. 7 illustrates an embodiment of an optic that does not include echelettes that repeat having the same width in r-squared space. The optic includes a diffractive profile 700 including a plurality of echelettes, each echelette of the diffractive profile 700 having a different width in r-squared space than any other echelette of the diffractive profile 700, and each echelette of the diffractive profile 700 being configured to distribute light to a distance focus. The diffractive profile 700 may serve to provide for an extended range of vision that may help to improve the visual performance at intermediate distances and may reduce other visual symptoms associated with diffractive optics including glare and halos.

In the embodiment of FIG. 7, a diffractive profile 700 is provided that includes a plurality of echelettes 702a-d and that is disposed on a surface of an optic such that each one of the plurality of echelettes 702a-d have a different width than each other in r-squared space. The diffractive profile 700 is shown relative to the Y axis 706, which represents the phase shift of the diffractive profile 700. The height is shown in units of millimeter (mm), and may represent the distance from the base spherical wavefront generated by the lens. In other embodiments, other units or scalings may be utilized. The height or phase shift of the diffractive profile 700 is shown in relation to the radius on the X axis 708 from the optical axis 704 in r-squared space. The radial coordinate represents the distance from the optical axis 704 in r-squared space, and is shown in units of millimeters squared, although in other embodiments, other units or scalings may be utilized.

Each echelette 702a-d may be positioned on a surface of the optic, with the surface extending radially outward from the optical axis 704 to the outer periphery of the optic (such as the outer periphery 27 marked in FIG. 2B). Each echelette 702a-d may be positioned adjacent to each other, as shown in FIG. 7, or in other embodiments spacings may be provided between the echelettes 702a-d.

The different widths in r-squared space of the echelettes 702a-d may correspond to different add powers of the optic. The echelette 702b for example, may direct light to a focus at an add power of 3 diopter, for example. The echelette 702c for example, may direct light to a focus at an add power of 2.4 diopter, for example. The echelette 702d for example, may direct light to a focus at an add power of 1.8 diopter, for example. The add power may be different for each echelette 702a-d. Various other add powers may be utilized as desired.

The echelettes 702a-d may each distribute some light to a distance focus, to provide a smooth, extended depth of focus or extended range of vision for the individual, decreased visual symptoms, and improved distance vision. Further, the echelettes 702a-b may each distribute some light to a near focus. The echelettes 702a-d may be configured to split light to both a distance focus and a near focus.

The irregular width of the echelettes 702a-d may provide a smooth, extended depth of focus or extended range of vision for the individual and may break the symmetry between the r-squared distances of transition zones between the echelettes, thus reducing undesired visual symptoms.

One or more of the echelettes 702a-d may have the same step height as each other. For example, as shown in FIG. 7, the echelettes 702b-d may have the same step heights as each other. In other embodiments, any or at least two of the echelettes 702a-d may have a same step height or a different step height than each other.

In one embodiment, the diffractive profile 700 may include a plurality of echelettes 702a -d disposed on a surface of the optic such that each one of the plurality of echelettes 702a-d between the optical axis and the outer periphery of the optic has a different width in r-squared space than any other echelette on the surface of the optic between the optical axis and the outer periphery of the optic.

In one embodiment, the echelettes 702a-d of the diffractive profile 700 may be positioned in a central zone 710, that the optical axis 704 may pass through. The optic may include a peripheral zone 712 positioned radially outward of the central zone 710, which may not include a diffractive profile in certain embodiments. The peripheral zone 712 as shown in FIG. 7 may include a refractive surface, which may be adjacent to the central zone 710 and the diffractive profile 700. Other configurations of optic may be utilized, for example, the diffractive profile 700 may extend outward to the outer periphery of the optic in certain embodiments, or a central zone 710 may not include a diffractive profile, with a peripheral zone including a diffractive profile, or an intermediate zone (between refractive surfaces) including a diffractive profile.

The number of echelettes of the diffractive profile 700 may vary. For example, the number may include at least three echelettes. The number may include at least four echelettes. The number may include at least five echelettes. A greater or lesser number of echelettes may be utilized as desired. In each embodiment, a step height of at least two of the echelettes may be the same, or a step height of at least two of the echelettes may be different.

An optic for an ophthalmic lens that includes a diffractive profile disclosed herein may be fabricated utilizing a variety of methods. A method may include determining optical aberrations of a patient's eye. Measurements of a patient's eye may be made in a clinical setting, such as by an optometrist, ophthalmologist, or other medical or optical professional. The measurements may be made via manifest refraction, autorefraction, tomography, or a combination of these methods or other measurement methods. The optical aberrations of the patient's eye may be determined.

A determination of the visual range of the patient may also be determined. For example, the ability of the patient to focus on near objects (presbyopia) may be measured and determined. A range of add power for the ophthalmic lens may be determined.

The measurements of the patient's eye may be placed in an ophthalmic lens prescription, which includes features of an optic that are intended to address the optical aberrations of the patient's eye, as well as features that address the visual range for the patient (e.g., an amount of add power and number of focuses to be provided by the optic).

The ophthalmic lens prescription may be utilized to fabricate an optic for the ophthalmic lens. A refractive profile of the optic may be determined based on the ophthalmic lens prescription, to correct for the optical aberrations of the patient's eye. Such a refractive profile may be applied to the optic, whether on a surface including the diffractive profile or on an opposite optical surface. The diffractive profile may also be determined to provide for the desired distribution of add power for the optic.

The determination of one or more of a refractive or diffractive profile and the fabrication of the optic may be performed remotely from the optometrist, ophthalmologist, or other medical or optical professional that performed the measurements of a patient's eye, or may be performed in the same clinical facility of such an individual. If performed remotely, the fabricated optic may be delivered to an optometrist, ophthalmologist, or other medical or optical professional, for being provided to a patient. For an intraocular lens, the fabricated optic may be provided for implant into a patient's eye.

The fabricated optic may be a custom optic fabricated specifically for the patient's eye, or may be fabricated in a manufacturing assembly and then selected by an optometrist, ophthalmologist, or other medical or optical professional for supply to a patient, which may include implantation in the patient's eye.

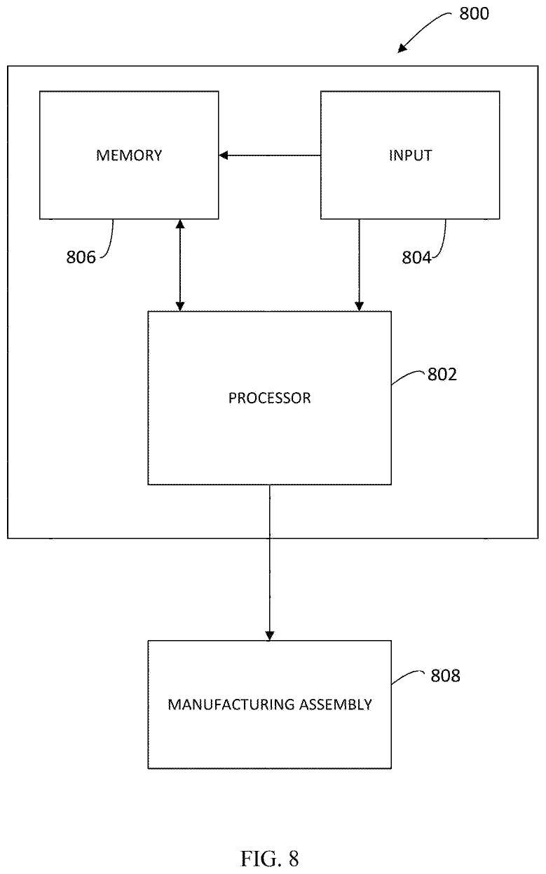

FIG. 8 illustrates an embodiment of a system 800 that may be utilized to perform all or a portion of the methods disclosed herein. The system 800 may include a processor 802, an input 804, and a memory 806. In certain embodiments the system 800 may include a manufacturing assembly 808.

The processor 802 may comprise a central processing unit (CPU) or other form of processor. In certain embodiments the processor 802 may comprise one or more processors. The processor 802 may include one or more processors that are distributed in certain embodiments, for example, the processor 802 may be positioned remote from other components of the system 800 or may be utilized in a cloud computing environment. The memory 806 may comprise a memory that is readable by the processor 802. The memory 806 may store instructions, or features of intraocular lenses, or other parameters that may be utilized by the processor 802 to perform the methods disclosed herein. The memory 806 may comprise a hard disk, read-only memory (ROM), random access memory (RAM) or other form of non-transient medium for storing data. The input 804 may comprise a port, terminal, physical input device, or other form of input. The port or terminal may comprise a physical port or terminal or an electronic port or terminal. The port may comprise a wired or wireless communication device in certain embodiments. The physical input device may comprise a keyboard, touchscreen, keypad, pointer device, or other form of physical input device. The input 804 may be configured to provide an input to the processor 802.

The system 800 may be utilized to perform the methods disclosed herein, such as the processes of determining a diffractive profile of the optic, as well as a refractive profile of the optic. The processor 802 may be configured to determine the diffractive profile to provide for various add powers for the optic, as well as determining a refractive profile to correct for ocular aberrations of the patient.

The processor 802 may provide the refractive profile and/or diffractive profile to the manufacturing assembly 808, which may be configured to fabricate the optic for the ophthalmic lens based on the refractive profile and/or diffractive profile. The manufacturing assembly 808 may comprise one or more apparatuses for forming the optic, and may comprise a high volume manufacturing assembly or a low volume manufacturing assembly. The manufacturing assembly 808 may be used for manufacture remote to a clinic in which measurements of the individual's eye or made, or local to such a clinic. The manufacturing assembly may include apparatuses such as lathe tools, or other lens formation devices to fabricate the optic.

In one embodiment, the processor 802 may be provided with an ophthalmic lens prescription for the individual's eye that may be provided as discussed herein. The processor 802 may receive the ophthalmic lens via the input 804. The system 800 may fabricate the optic for the ophthalmic lens based on the prescription.

The system 800 may be configured to fabricate any of the embodiments of ophthalmic lenses disclosed herein.

In one embodiment, a diffractive profile such as the profile 700 may be positioned on a surface of a lens that is opposite an aspheric surface. The aspheric surface on the opposite side of the lens may be designed to reduce corneal spherical aberration of the patient.

In one embodiment, one or both surfaces of the lens may be aspherical, or include a refractive surface designed to extend the depth of focus, or create multifocality.

In one embodiment, a refractive zone on one or both surfaces of the lens may be utilized that may be the same size or different in size as one of the diffractive zones. The refractive zone includes a refractive surface designed to extend the depth of focus, or create multifocality.

Any of the embodiments of lens profiles discussed herein may be apodized to produce a desired result. The apodization may result in the step heights and step offsets of the echelettes being gradually varied according to the apodization, as to gradually increasing the amount of light in the distance focus as a function of pupil diameter.

The features of the optics disclosed herein may be utilized by themselves, or in combination with refractive profiles of the optics and/or with features providing for correction of chromatic aberrations (e.g., achromats, which may be diffractive).

The ophthalmic lenses disclosed herein in the form of intraocular lenses are not limited to lenses for placement in the individual's capsular bag. For example, the intraocular lenses may comprise those positioned within the anterior chamber of the eye. In certain embodiments the intraocular lenses may comprise “piggy back” lenses or other forms of supplemental intraocular lenses.

Features of embodiments may be modified, substituted, excluded, or combined as desired.

In addition, the methods herein are not limited to the methods specifically described, and may include methods of utilizing the systems and apparatuses disclosed herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method comprising:
fabricating an optic for an ophthalmic lens, the optic including a diffractive profile including a plurality of echelettes, each echelette of the diffractive profile having a different width in r-squared space than any other echelette of the diffractive profile and each echelette of the diffractive profile being configured to distribute light to a distance focus.

2. The method of claim 1, further comprising receiving an ophthalmic lens prescription, and fabricating the optic based on the ophthalmic lens prescription.

3. The method of claim 2, further comprising determining one or more of the diffractive profile or a refractive profile of the optic based on the ophthalmic lens prescription.

4. The method of claim 1, wherein the plurality of echelettes include at least 3 echelettes.

5. The method of claim 1, wherein the diffractive profile forms an extended depth of focus.

6. A system for fabricating an ophthalmic lens, the system comprising:
a processor configured to determine a diffractive profile of an optic, the diffractive profile including a plurality of echelettes, each echelette of the diffractive profile having a different width in r-squared space than any other echelette of the diffractive profile and each echelette of the diffractive profile being configured to distribute light to a distance focus; and
a manufacturing assembly that fabricates the optic based on the diffractive profile.

7. The system of claim 6, further comprising an input for receiving an ophthalmic lens prescription, and wherein the processor is configured to determine one or more of the diffractive profile or a refractive profile of the optic based on the ophthalmic lens prescription.

8. The system of claim 6, wherein the plurality of echelettes include at least 3 echelettes.

9. The system of claim 6, wherein the diffractive profile forms an extended depth of focus for the optic.

10. The system of claim 6, wherein each echelette of the diffractive profile is configured to distribute light to a near focus.
